(19) United States
(12) Patent Application Publication
(10) Pub. No.: US 2013/0122396 A1
(43) Pub. Date: May 16, 2013

(54) METHOD AND DEVICE USING PLASMON- RESONATING NANOPARTICLES

(71) Applicants: Suljo Linic, Ann Arbor, MI (US); Phillip N. Christopher, Riverside, CA (US)

(72) Inventors: Suljo Linic, Ann Arbor, MI (US); Phillip N. Christopher, Riverside, CA (US)

(73) Assignee: THE REGENTS OF THE UNIVERSITY OF MICHIGAN, Ann Arbor, MI (US)

(21) Appl. No.: 13/696,763

(22) PCT Filed: May 19, 2011
(86) PCT No.: PCT/US2011/037148

§ 371 Date: Jan. 22, 2013

Related U.S. Application Data
(60) Provisional application No. 61/346,771, filed on May 20, 2010.

(51) Int. Cl. B01J 19/12; H01M 4/86; C07D 301/08; C01B 31/20; C01B 21/36; H01M 8/10; C07D 301/10

(52) U.S. Cl. CPC B01J 19/122; H01M 8/10; H01M 4/86; C07D 301/10; C01B 31/20; C01B 21/36; C07D 301/08
USPC 429/482; 549/534; 423/437.2; 423/403; 549/523; 422/186

(57) ABSTRACT

Disclosed herein are methods and articles that include a plasmon-resonating nanostructure that employ a photo-thermal mechanism to catalyze the reduction of an oxidant. As such, the plasmon-resonating nanostructure catalyzes a redox reaction at a temperature below a predetermined activation temperature. The method can be efficiently used to catalyze the reduction of an oxidant, for example in a catalytic reactor or in a fuel cell that includes a photon source.

### CROSS-REFERENCE TO RELATED APPLICATION

The benefit of priority under 35 USC §119(e) of U.S. provisional patent application Ser. No. 61/346,771 filed May 20, 2010, the disclosure of which is incorporated herein by reference, is claimed.

### STATEMENT OF GOVERNMENT INTEREST

This invention was made with governmental support under grants from the U.S. Department of Energy, Office of Basic Energy Sciences (DOE-BES) and Division of Chemical Science (FG-02-05ER15686), and the National Science Foundation (CBET 0756255). The government has certain rights in the invention.

### BACKGROUND

Industrial heteroegeneous catalytic processes are almost exclusively thermally activated. The catalytic process is driven by overcoming activation barriers or shifting equilibria by raising the temperature of the heterogeneous catalyst. The activation temperature of the catalytic process is typically the temperature necessary to overcome the highest activation barrier. These thermochemical processes are well understood, widely studied and have produced a massive library of catalytic materials that are suitable for the production of an immense range of products.

Despite their wide acceptance, thermochemical processes, commonly run at temperatures between about 200° C. to about 800° C., are extremely energy intensive, and are inherently difficult to finely control. For example, the design of optimal catalytic materials is hampered by the materials often changing size and/or shape upon heat which effects the catalytic activity/selectivity at high temperatures. Of course, one method to reduce the energy needs of the catalytic process and achieve fine control is reducing the operating temperature of the thermochemical process. A lower temperature limits changes to the size and shape of the catalytic particles. Naturally, this means that the activation temperature needs to be lower and the catalytic sites must have reasonable turn-over frequencies at the new lower temperatures.

Photocatalytic processes are typically less industrially applicable and almost exclusively require semiconducting materials. The materials must meet several strict requirements: electronic structures where the light excitation promotes the formation of electron/hole (e⁻/h) pairs, conduction bands at potentials greater than the reduction potential of the oxidant, valence bands at potentials less than the oxidation potential of the reductant, and e⁻/h lifetimes having durations sufficient to facilitate chemical reactions. These requirements are difficult to meet and, therefore, these materials are not considered to be sufficiently industrially versatile.

Despite the breadth of knowledge on the thermochemical processes, the prior art does not teach heterogeneous thermochemical catalytic processes promoted by photoexcitation (herein, photo-thermal catalysis). Moreover, the prior art fails to teach catalytic materials that combine thermocatalytic capabilities of metal catalysts with photochemical excitation.

### SUMMARY OF THE INVENTION

Disclosed herein are methods and articles that include a plasmon-resonating nanostructure employing a photo-thermal mechanism to catalyze the reduction of an oxidant. Generally, the plasmon-resonating nanostructure catalyzes a redox reaction at a temperature below a predetermined activation temperature. The plasmon-resonating nanostructure can be a nanoparticle that comprises copper, silver, gold or alloys thereof. The method can be efficiently used to catalyze the reduction of an oxidant, for example, in a catalytic reactor or in a fuel cell.

According to one embodiment, the method includes supplying an oxidant, having a π-antibonding orbital, to a surface of a plasmon-resonating nanostructure, exposing the nanostructure to photons at a wavelength sufficient to photoexcite the nanostructure, and reducing the oxidant at a rate that is about 1.1 to about 10,000 times the rate of reduction of the oxidant under the same conditions but in the absence of the photons.

According to another embodiment, the method includes supplying an oxidant, having a π-antibonding orbital, to a surface of a plasmon-resonating nanostructure, exposing the nanostructure to photons at a wavelength sufficient to photoexcite the nanostructure, and reducing the oxidant at a temperature below a predetermined thermodynamic barrier (such as, an activation temperature).

Various additional embodiments of the method may further include supplying a reductant, such as an alkene. The alkene can be selected from ethylene, propylene and butylene. Alternatively, the reductant can be selected from hydrogen, methanol, and ammonia. The plasmon-resonating nanostructure may be present on a support, for example silica and/or alumina. The method may further include producing an oxidation product, such as, for example water, ethylene oxide, propylene oxide, acrylonitrile, acrolein, acrylic acid, carbon dioxide, nitrous oxide, nitric oxide, nitrogen dioxide, and mixtures thereof. The oxidant can be dioxygen, dinitrogen, nitrous oxide and/or ozone; preferably, however, the oxidant is dioxygen.

Preferably, the plasmon-resonating nanostructure catalyzes the reduction of the oxidant. The plasmon-resonating nanostructure includes a nanoparticle that comprises copper, silver, gold or alloys thereof. The temperature at which the oxidant is reduced can be about 20 to about 100° C. below the predetermined activation temperature. The predetermined activation temperature is a temperature at which the plasmon-resonating nanostructure catalyzes the reduction of the oxidant in the absence of photons.

Yet another embodiment is an electrochemical cell that includes an electrolyte, a cathode that includes a plasmon-resonating nanostructure, an anode separated from the cathode by the electrolyte, and a photon-transfer device that is sufficiently transparent at a wavelength that photoexcites the plasmon-resonating nanostructure.

The cell can also include an oxidant in fluid communication with the cathode, a reductant in fluid communication with the anode, and an external circuit electrically connected to the cathode and to the anode. The electrolyte can be a polymer electrolyte membrane, which itself can be perfluorosulfonic acid polymer membranes, fluorosulfonic acid polymer membranes, sulfonated polymer membranes, acid-base complex membranes, ionic liquid based membranes, inorganic composite membranes, and mixtures thereof. The oxidant can be dioxygen and the reductant can be either hydrogen or methanol.

Still another embodiment is a device that includes a plasmon-resonating nanostructure, a support for the plasmon-resonating nanostructure, and a photon-transfer device that is sufficiently transparent at a wavelength that photoexcites the nanostructure.

The device can include an oxidant and a reductant in fluid communication with the plasmon-resonating nanostructure. The oxidant can be dioxygen and the reductant can be ethylene.

Additional features of the invention may become apparent to those skilled in the art from a review of the following detailed description, taken in conjunction with the drawings, the examples, and the appended claims.

### BRIEF DESCRIPTION OF THE DRAWINGS

For a more complete understanding of the disclosure, reference should be made to the following detailed description and accompanying drawing figures wherein:

FIG. 1 is a plot of the mass spectroscopy signal at a M/z=44 for ethylene epoxidation over a silver nanoparticle catalysts at 180° C., wherein the silver nanoparticle catalysts was exposed to light at about 950 seconds and the light was removed off at about 1800 seconds;

FIG. 2 is a comparison plot showing the photo enhancement, thermal activity and photothermal activity as a function of reaction temperature for the epoxidation of ethylene;

FIG. 3 is a plot comparing the reaction kinetics for the thermal and photothermal pathways for the epoxiation of ethylene;

FIG. 4 is a plot of the ethylene oxide selectivity as a function of temperature for thermal and photothermal processes;

FIG. 5 is a plot showing the yield enhancement as a function of temperature for the photothermal epoxidation of ethylene;

FIG. 6 is a comparison plot of the rate enhancement and thermal and photo production rates for CO oxidation;

FIG. 7 is a comparison plot of the rate enhancement and thermal and photothermal production rates for NH₃ oxidation;

FIG. 8 is Steady state product production for O¹⁶ based process (red squares) and O¹⁸ based process (blue squares) for ethylene epoxidation;

FIG. 9 is a proposed mechanism of photo-enhancement, where plasmons decay into energetic electrons (energy 2-3 eV above the silver Fermi level) and can transfer into the antibonding orbital of O₂ adsorbed on the silver surface;

FIGS. 10, 11, and 12 are graphical results, run 1, showing data corresponding to the oxidation of ethylene with dioxygen at 125, 150, 170, 190, 200, 210, and 220° C.;

FIGS. 13, 14, and 15 are graphical results, run 2, showing data corresponding to the oxidation of ethylene with dioxygen 150, 170, 190, 200, 210, 220, and 230° C.;

FIG. 16 is a plot of data from runs 1 and 2 comparing the activity enhancement as a function of temperature;

FIG. 17 is a drawing of a direct methanol fuel cell;

FIG. 18 is a drawing of a fuel cell;

FIG. 19 is a graphical representation of data collected from the oxidation of CO with dioxygen at 170° C. showing the enhancement of the catalytic oxidation after the plasmon-resonating nanoparticles are exposed to light (a photon source);

FIG. 20 is a drawing of a catalytic reactor or device;

FIG. 21 is a drawing of a catalytic reactor or device having a thin film of the plasmon-resonating nanoparticles and conduits for supplying and removing oxidant and reductants from the plasmon-resonating nanoparticles. The plasmon-resonating nanoparticles were exposed to the sun as a photon source;

FIG. 22 is a plot of the photo-rate enhancement for CO oxidation (circles) and NH₃ oxidation (squares) as a function of temperature;

FIG. 23 (top) is a plot of the thermal (squares) and photo-thermal (circles) reaction rates for propylene epoxidation over a 2% Cu/SiO₂ catalyst; (bottom) is a plot of the rate enhancement based on the photo-thermal propylene epoxidation over a 2% Cu/SiO₂ catalyst;

FIG. 24 is a plot of the selectivity for thermal and photo-thermal propylene epoxidation over 2% Cu/SiO₂ catalyst vs. the reaction rate where the two major products are propylene oxide and acrolein;

FIG. 25(a) is a normalized plot of (blue circles) the rate of ethylene epoxidation at 470 K as a function of filter cutoff wavelength and (red squares) the plasmon intensity of the silver catalyst of the ethylene epoxidation at 470 K as a function of filter cut off wavelength;

FIG. 26 is a plot of the rate of ethylene epoxidation vs. incident photon intensity as a function of reactor temperature;

FIG. 27 is a plot of the conversion efficiency for ethylene epoxidation vs. temperature as a function of incident photon intensity; and,

FIG. 28 is a plot of the conversion efficiency per quantum of light (reported as a quantum efficiency) vs. temperature as a function of incident photon intensity.

While the disclosed compositions, method and apparatus are susceptible of embodiments in various forms, there are illustrated in the examples and figures (and will hereafter be described) specific embodiments, with the understanding that the disclosure is intended to be illustrative and is not intended to limit the invention to the specific embodiments described and illustrated herein.

### DETAILED DESCRIPTION

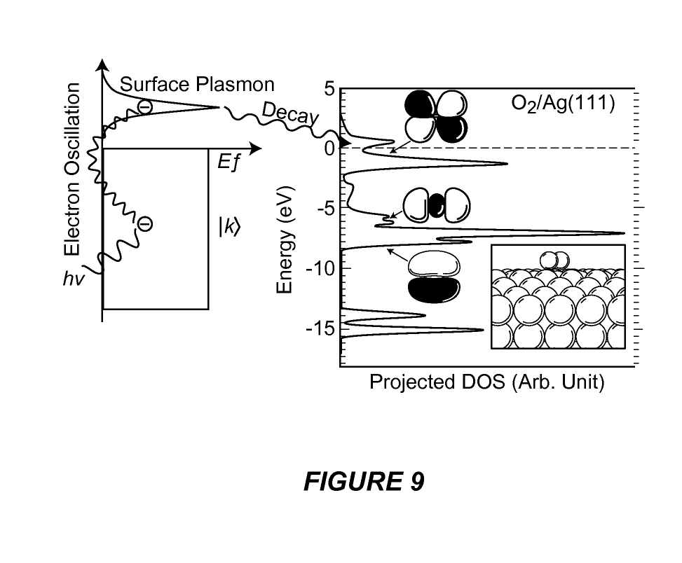

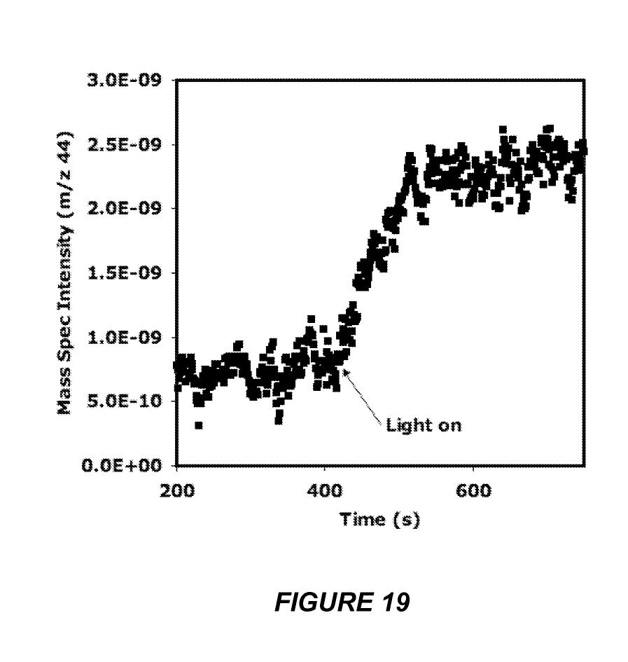

Herein are described methods and apparatus with catalytic materials that combine thermocatalytic reactions with surface plasmon light concentration to enhance catalytic activity. This combination of mechanistic pathways effectively lowers the operating temperature necessary for a desired product yield by a substantial amount, e.g., 20° C. to 100° C. for un-optimized reactor geometries. Exemplary of the methods and devices described herein, examples described herein indicate that visible light illumination of copper or silver nanoparticles produces a strong plasmon resonance and allows for the efficient transfer of excited electrons from the nanoparticles to dioxygen (O₂) anti-bonding orbitals (see, e.g., FIG. 9). This combined photochemical-thermochemical (photo-thermal) method effectively decreases the thermal energy necessary to traverse the activation barrier for the rate limiting step of oxidation reactions, e.g., over silver catalysts (O₂ reduction). See FIG. 19. Preferably, this photo-thermal method effectively increases the rate at which the oxidant can be reduced as compared to the same conditions in the absence of photons, i.e., a pure thermal method. For example, the rate of reduction of the oxidant can be about 1.1, 1.2, 1.3, 1.4, 1.5, 1.6, 1.7, 1.8, 1.9, 2, 10, 50 to about 2.5, 5, 10, 15, 20, 25, 30, 35, 50, 100, 500, 1,000, or 10,000 times the rate of reduction of the oxidant under the same conditions but in the absence of the photons, including reducing the oxidant at a rate about 1.1 to about 10,000, about 10 to about 1,000, or about 50 to about 500 times the rate of reduction of the oxidant under the same conditions but in the absence of the photons.

As used herein, “nanostructure” generally refers to a particle that exhibits one or more properties not normally associated with a corresponding bulk material (e.g., quantum optical effects). The term also generally refers to materials having at least two dimensions that do not exceed about 1000 nm. In various embodiments described herein, these dimensions are even smaller.

The methods, cells and devices, herein, include a plurality of nanostructures, that is a plurality of individual nanostructures; alternatively, a plurality of differing individual nanostructures. Herein, a nanostructure includes one or more nanoparticles or nanocrystals, e.g., a nanostructure can be a single nanoparticle or a plurality of adhered nanoparticles. Nanostructure does not refer to a macroscale structure that may include nanostructures. Preferably, a nanostructure including a plurality of nanoparticles has a structure where the nanoparticles are adhered to one another to form a single particle with nanometer scale dimensions. Herein, nanoparticles and nanocrystals are synonymous and refer to submicron (nanometer) sized materials with a crystalline structure, the nanoparticles and nanocrystals can have a variety of shapes, dependent or independent, on the crystalline structure. As is understood by those of ordinary skill in the art, the term nanoparticles refers explicitly to a crystalline material, herein preferably made of copper, silver, gold, or alloys thereof. The description of the size and/or shape of a nanoparticle refers to the crystalline material, typically determined by TEM.

Furthermore, a nanostructure can include a plurality of nanoparticles and nanocrystals of different sizes. In one embodiment, a nanostructure includes a “large” nanoparticle and one or more “small” nanoparticles having a different chemical formulation than the “large” nanoparticle. Preferably, the “small” nanoparticle has an effective diameter less than 50%, 45%, 40%, 35%, 30%, 25%, 20%, 15%, 10%, and/or 5% of an effective diameter of the “large” nanoparticle.

Recent developments in solution-based techniques have allowed for the synthesis of nanoparticles with well-controlled, highly-uniform sizes, and particle geometries. Some of these nanoparticles (e.g., metals with free-electron-like valence bands, such as noble metals) exhibit a strong localized surface plasmon resonance (LSPR) due to the nanometer scale spatial confinement, and the metal's inherent electronic structure. For example, the resonance frequency of silver (Ag) nanoparticles falls in the ultraviolet to visible light range, and can be tuned by changing the geometry and size of the particles. The intensity of resonant electromagnetic radiation is enhanced by several orders of magnitude near the surface of plasmonic (or plasmon-resonating) nanoparticles. Disclosed herein are compositions that exploit the ability of plasmonic nanoparticles to create electron-hole (e⁻/h) pairs.

Plasmon resonance is an optical phenomenon arising from the collective oscillation of conduction electrons in a metal when the electrons are disturbed from their equilibrium positions. Such a disturbance can be induced by electromagnetic energy (light), in which the free electrons of a metal are driven by the alternating electric field to coherently oscillate at a resonant frequency relative to the lattice of positive ions. The plasmon frequencies for most metals occur in the UV region of the electromagnetic spectrum, with alkali metals and some transition metals, such as copper, silver, and gold, exhibiting plasmon frequencies in the visible region of that spectrum. A “plasmon-resonating” (or “plasmonic”) nanoparticle, therefore, is a nanoparticle having conduction electrons that collectively oscillate when disturbed from their equilibrium positions.

Herein, the plasmon-resonance of the plasmon-resonating nanostructure is induced by electromagnetic energy. Typically, this energy is delivered as photons from a light source, for example by exposing the plasmon-resonating nanostructure to photons emitted from a light source. As is well understood in the art, a photon is a discrete packet of energy or a unit of electromagnetic radiation, including light. The energy of the photon is described by E=hv and hv is often used as a designation of light or photonic energy. As used herein, the light source (photon source) is a object, structure, or device that emits, transmits, or generates electromagnetic energy. The photon source can be a laser, a lamp and/or the Sun. In embodiments where the electromagnetic energy is conducted or transmitted through a photon-transfer device from a photon-generating source, e.g., laser tube or the Sun, to the plasmon-resonating nanostructure, the photon source is understood to be the point at which photons are emitted from the transfer device (e.g., fiber optic cable, mirror, lens, window and mixtures thereof). In embodiments where the photon-generating source is conducted or transferred through a photon-transfer device, the photon-transfer device is preferably sufficiently transparent at a wavelength that photoexcites the plasmon-resonating nanostructure, such that greater than 25% of the light entering the transfer device is transmitted or emitted from the photon-transfer device, that is, the transfer device has a percent transmission of greater than 25%. Preferably, the percent transmission is greater than 50%, 60%, 70%, 80%, or 90%.

The frequency and intensity of a plasmon resonance are generally determined by the intrinsic dielectric property of a given metal, the dielectric constant of the medium in contact with the metal, and the pattern of surface polarization. As such, any variation in the shape or size of a metal particle that can alter the surface polarization and causes a change to the plasmon resonance. This dependence offers the ability to tune the surface plasmon resonance, or localized surface plasmon resonance (LSPR) of metal nanoparticles through shape-controlled synthesis. Such synthesis are generally described in Lu et al. (2009) Annu. Rev. Phys. Chem. 60:167-92, the disclosure of which is incorporated herein by reference.

The plasmon-resonating nanostructure can have any shape, but generally and preferably has a shape that is spherical (nanospheres), cubic (nanocubes), or wire shape (nanowires). In a preferred embodiment, the plasmon-resonating nanostructures include nanoparticles that are cubic (nanocubes). The shapes of these plasmon-resonating nanostructures can be obtained by various nanoparticle synthesis methods such as, for example, those described in the U.S. Pat. No. 7,820,840 B2, incorporated herein by reference.

In each of these shapes, the plasmon-resonating nanostructure and nanoparticle will have an effective diameter, which as used herein is the smallest cross-section of the plasmon-resonating nanostructure or the plasmon-resonating portion thereof, e.g., a plasmon-resonating nanoparticle or a plasmon-resonating layer. Thus, for example, the effective diameter of a plasmon-resonating nanowire is determined based on the smallest cross-section of the nanowire, for example, as measured by TEM. Further, the effective diameter of a plasmon-resonating nanosphere will coincide with and be the same as the diameter of the nanosphere. Generally, the plasmon-resonating nanostructures should have an effective diameter of about 10, 20, 30, 40, 50, 60, 70, 80, 90, 100 nm to about 100, 110, 120, 130, 140, 150, 160, 170, 180, 190, 200 nm, preferably about 30 nm to about 170 nm, more preferably about 30 nm to about 100 nm. In the context of a plasmon-resonating nanocube, the nanocube will have an effective diameter coincident with the cube edge-length and of about 10 nm to about 200 nm; preferably about 90 nm to about 150 nm. Generally, the wavelength of light plasmon-resonated by the nanostructure will vary with the size and shape of the nanostructures. For example, the larger the plasmon-resonating nanostructure within these ranges, the greater the wavelength of light affected.

In various embodiments, the light applied to the nanostructure can be ultraviolet light (10 nm to 380 nm), visible light (380 nm to 780 nm), or infrared light (780 nm to 1000 gm). The light, that is the wavelengths of the photons to which the plasmon-resonating nanostructure is exposed, can be full spectrum or curtailed by filters or by function of the photon source (e.g., lasers are typically simple wavelength sources).

In various embodiments, the plasmon-resonating nanostructures include at least one of copper, silver, and gold nanoparticles. These nanoparticles may be copper/silver/gold alloy nanoparticles (e.g., copper-silver nanoparticles, copper-gold nanoparticles, silver-gold nanoparticles, copper-silver-gold nanoparticles). The nanostructures also may include, for example, silica as a core onto which the copper, silver and/or gold are deposited. In another variation, the nanostructures can be particles of substrates, for example silica, platinum, or other metal particles, onto which a plasmon-resonating layer or plasmon-resonating nanoparticle is deposited, e.g., layers or nanoparticles of Cu, Ag, and/or Au. In one preferred embodiment, the nanostructures include copper. In another preferred embodiment, the nanostructures include silver. In yet another preferred embodiment, the nanostructures include gold.

In another embodiment, a nanostructure that includes a “large” nanoparticle and a “small” nanoparticle includes a first nanoparticle that is a copper, silver, and/or gold nanoparticle and a second nanoparticle having a different chemical formulation than the first nanoparticle. The first nanoparticle can be either the large or the small nanoparticle, likewise the second nanoparticle can be either the small or the large nanoparticle. The second nanoparticle includes thermocatalysts known in the art. Specific examples include, but should not be limited to platinum, palladium, ruthenium, nickel, iron, and alloys thereof. One such example includes a “large” silver nanocube and a plurality of “small” platinum nanoparticles adhered to the silver nanocube. Preferably, the nanostructure includes a first nanoparticle selected from the group consisting of copper, silver, and gold nanoparticles, and a plurality of a second nanoparticle adhered to the first nanoparticle. In one embodiment, a nanostructure including a “large” nanoparticle and a plurality of “small” nanoparticles is produced by a dispersing the “large” nanoparticle in a solution containing a soluble precursor to the “small” nanoparticle, then selectively reducing the soluble precursor to deposit “small” nanoparticles on the “large” nanoparticle. An similar method was reported by Lim, et al. “Pd-Pt Bimetallic Nanodendrites with High Activity for Oxygen Reduction” Science, 324, 1302-1305 (2009), the method incorporated herein by reference.

Preferably, the plasmon-resonating nanostructure interacts with an oxidant. The oxidant can be supplied to the nanostructure in any available form, e.g., as a gas, liquid or mixture, in a flow through or static reactor. As used herein, the term oxidant refers to a chemical species that is capable of being reduced by a sufficiently energetic electron. In one embodiment, the oxidant is selected from dioxygen (O₂), dinitrogen (N₂), nitrous oxide (N₂O), ozone (O₃), and mixtures thereof. Preferably, the oxidant is dioxygen. Typically, when the oxidant is dioxygen, the oxidant is supplied as a mixture with a gas or liquid that is not functioning as an oxidant. For example, dioxygen can be a mixture with dinitrogen (e.g., air). As stated above, the function of the chemical species as an oxidant is dependant on the potential of the reducing electron.

Often an oxidant is used to oxidize a reductant. As used herein, the term reductant refers to a chemical species that is capable of providing an electron. In one embodiment, the reductant is selected from an alkene (e.g., ethylene, propylene, butylene (including 1-butylene, 2-butylene and isobutylene)), hydrogen, methanol, and ammonia. While the reductant can be a mixture, the reductant is preferably a single chemical species.

The redox chemistries of the reductant and oxidant can produce an oxidation product selected from a group consisting of water, ethylene oxide, propylene oxide, acrylonitrile, propenal, acrylic acid, carbon dioxide, nitrous oxide, nitric oxide, nitrogen dioxide, and mixtures thereof.

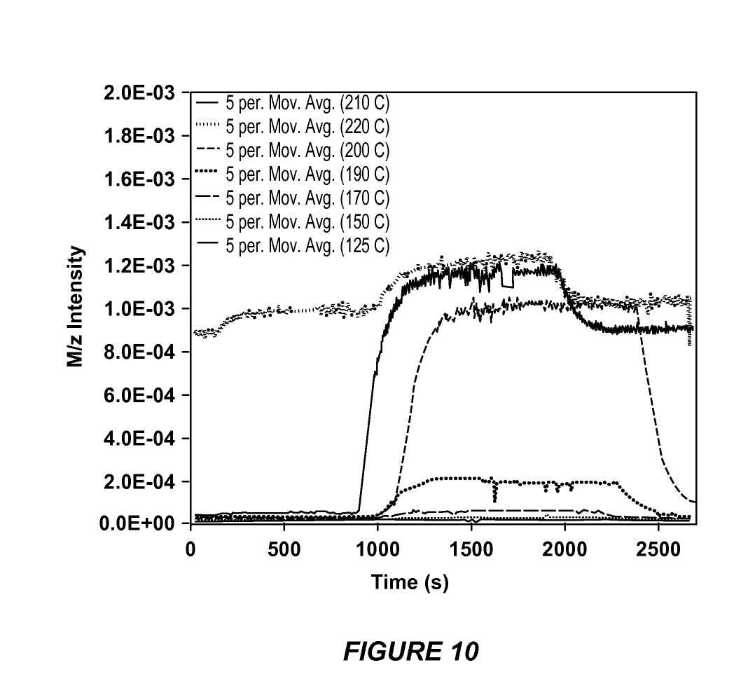

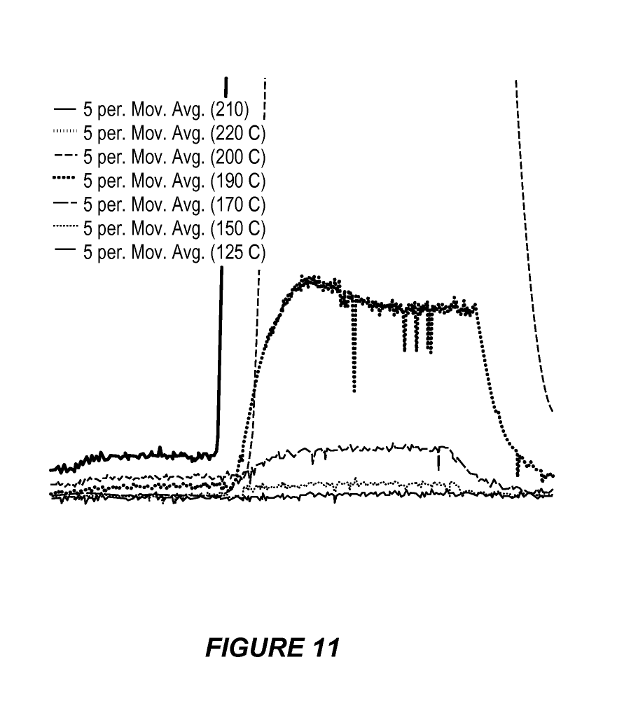

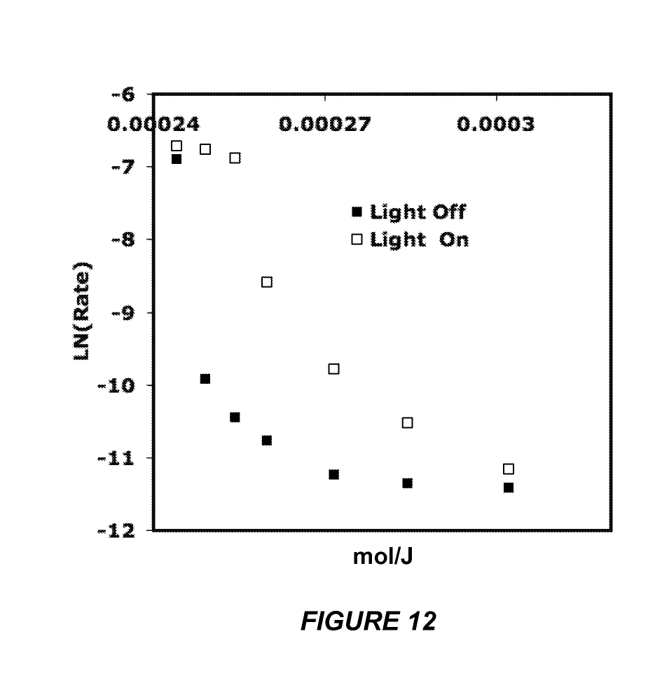

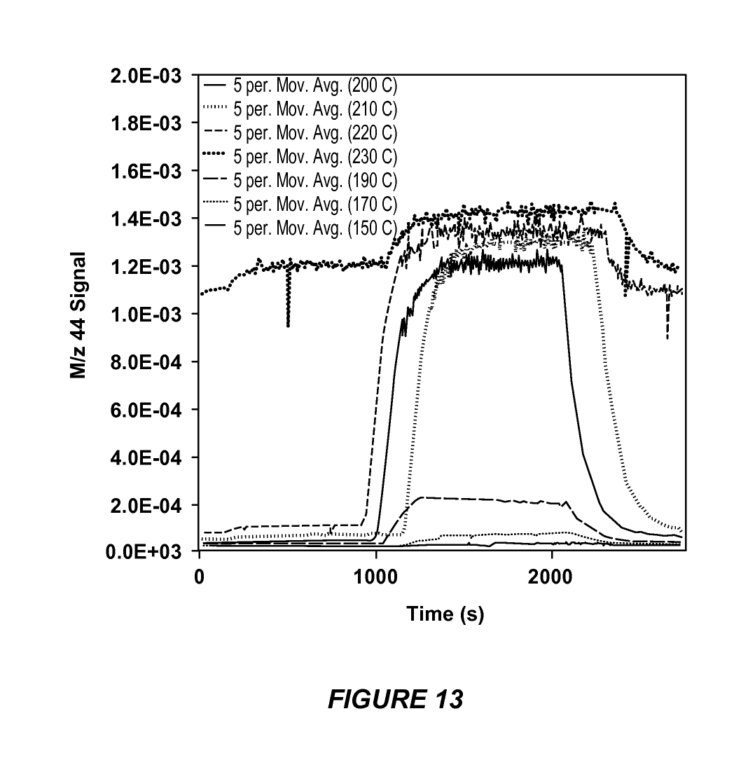

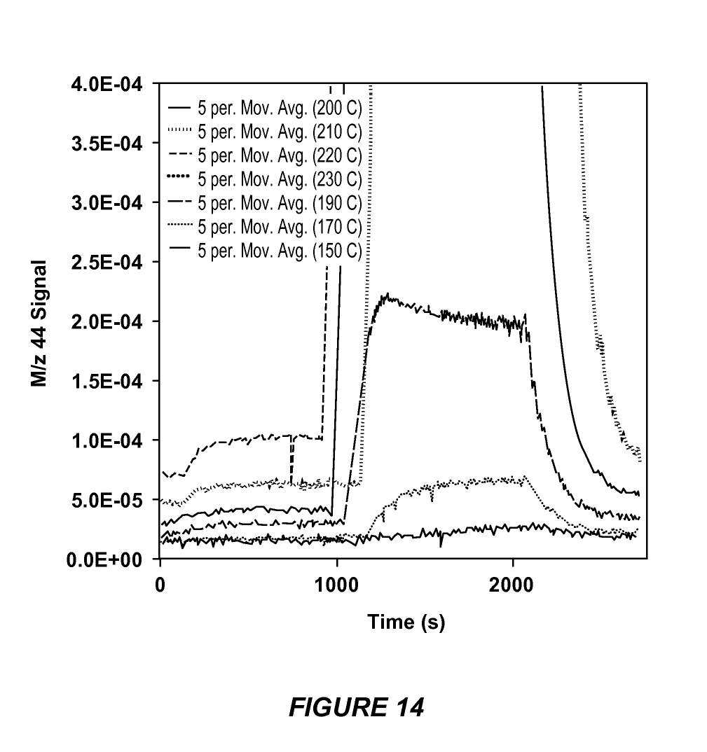

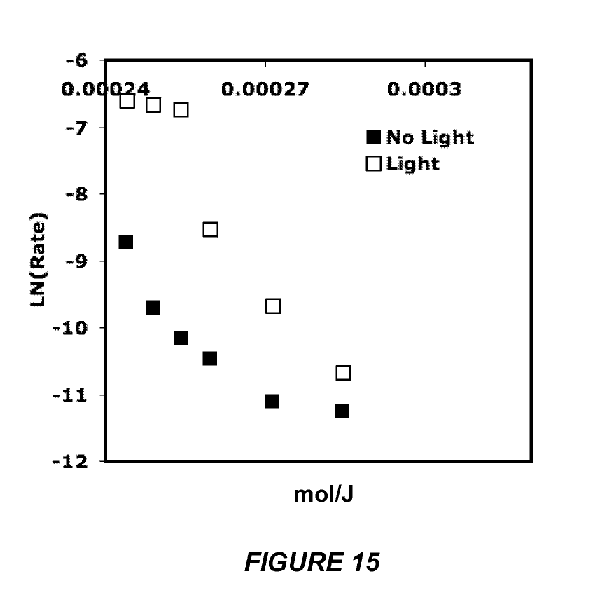

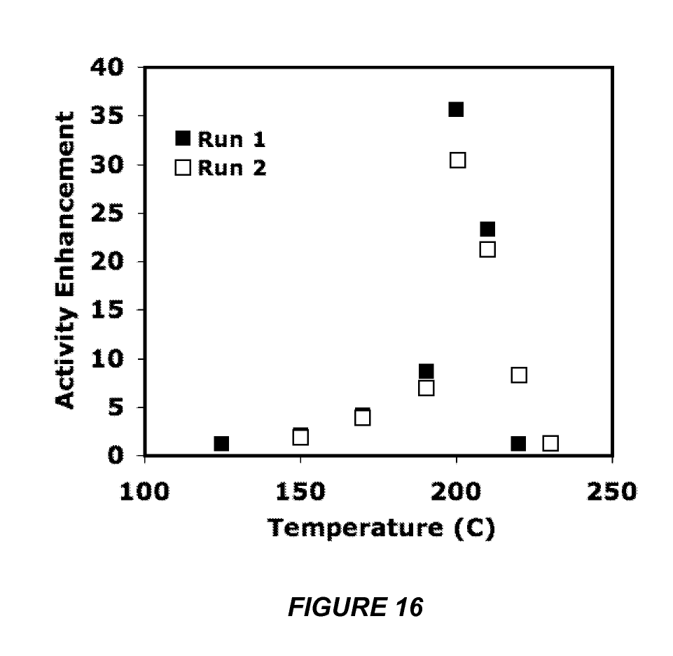

The redox chemistries (the oxidation of the reductant and reduction of the oxidant) may occur without the plasmon-resonating nanostructure but herein the plasmon-resonating nanostructure catalyses the reaction. One benefit of catalytic reactions is that the temperature necessary to drive the reaction can be decreased. For any specific reaction there is an activation temperature. As used herein “activation temperature” is the minimum temperature necessary to overcome a thermodynamic barrier in a reaction pathway. Often reaction rates scale with increasing temperature but the energy input necessary to overcome the thermodynamic barrier remains the same. Herein, the plasmon-resonating nanostructure may catalyze the reaction of an oxidant with a reductant at a predetermined activation temperature. When exposed to a light source, the photon influx upon the plasmon-resonating nanostructure allows for the reaction temperature to be decreased below the predetermined activation temperature, yielding a photo-thermal catalytic process. Preferably, the photo-thermal catalytic process for a specific plasmon-resonating nanostructure can be run (driven) at a temperature at least about 10° C., about 20° C., about 30° C., about 40° C., about 50° C., about 60° C., about 70° C., about 80° C., about 90° C., and/or about 100° C. below the predetermined activation temperature for that plasmon-resonating nanostructure, e.g. at a temperature in a range of about 20° C. to about 100° C., about 30° C. to about 90° C., about 40° C. to about 80° C. below the predetermined activation temperature. See FIGS. 10-16. Moreover and specifically for clarification, the predetermined activation temperature is a temperature at which the plasmon-resonating nanostructure catalyzes the reduction of the oxidant in the absence of the photons. Thereby, the predetermined activation temperature can be above the minimum temperature necessary to overcome the reaction's activation energy.

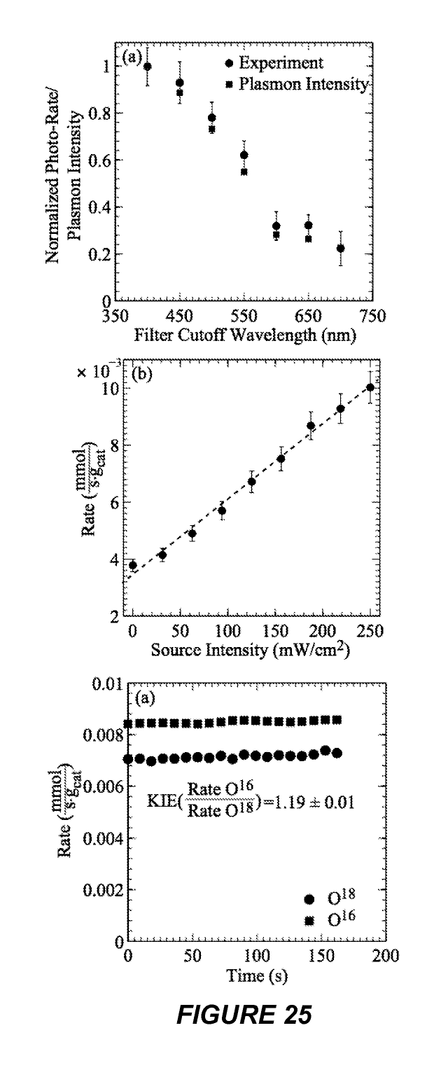

Without being bound to the theory, the mechanism of the herein described photo-catalytic activity is believed to be a result of an electron driven process mediated by plasmon excitation. FIG. 25(a) indicates a direct correlation between the wavelength dependence of the silver catalyzed photocatalytic activity for ethylene epoxidation and the wavelength dependence of the silver plasmon intensity. FIG. 25(b) indicates a direct (linear) correlation between the photocatalytic activity and the source intensity up to 250 mW/cm². This linear dependence on source intensity is indicative of an electron driven process. The dashed line shows a linear fit to the experimental data with the form: Rate=2.66*10⁻⁵ (intensity)+3.43*10⁻³. FIG. 25(c) shows the steady state rates for the photo-thermal reactions 450 K for ¹⁶O₂ (red squares) and ¹⁸O₂ (blue circles) reactants and shows the result of switching from ¹⁶O₂ (at least 99% ¹⁶O₂) to ¹⁸O₂ (at least 99% ¹⁸O₂) in the photo-thermal ethylene epoxidation with a silver catalyst; a 16% decrease in reaction rate. This comparison of the isotopic effect for the analogous thermal process showed a 5% decrease in reaction rate when ¹⁸O₂ was used. These results suggest that below a source intensity of 250 mW/cm² the reaction proceeds by a single electron process.

In another embodiment, the plasmon-resonating nanostructure can be on or carried by a support. Preferably the support is a non-conductive material, e.g., an insulator. More preferably, the support is thermally stable at the temperature at which the photo-thermal catalytic process is run. Moreover, the support is preferably sufficiently optically transparent to permit incident photo-irradiation to penetrate the substrate and interact with plasmon-resonating nanostructures below an outer surface. Examples of supports include but are not limited to silica, alumina, and mixtures thereof. Supports can further include polymers and polymeric material.

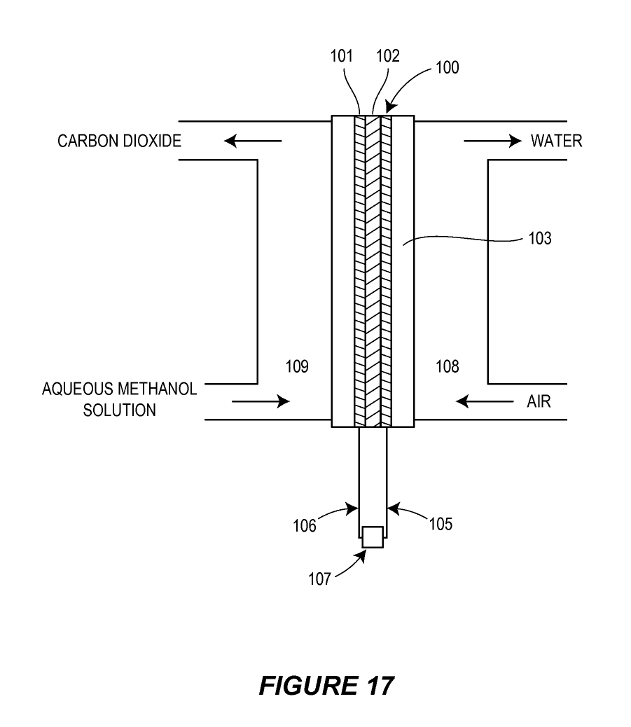

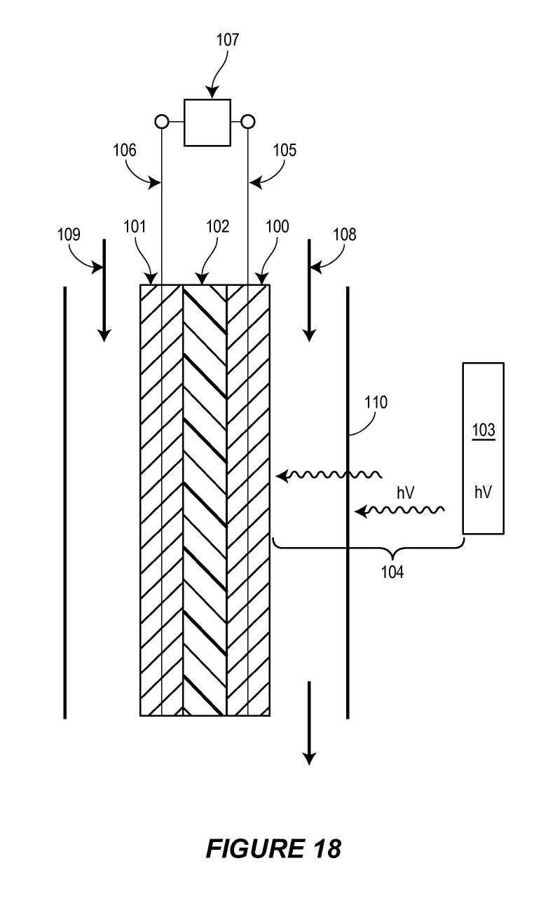

The plasmon-resonating nanostructure described herein can be used in an electrochemical cell. The electrochemical cell can have a fuel cell design or other applicable design wherein the photo-thermal catalytic process yields an electrical potential. FIGS. 17 and 18 depict basic fuel cell designs wherein a cathode 100 that includes a plasmon-resonating nanostructure is separated from an anode 101 by an electrolyte 102. The fuel cell design differs from those known in the art by the inclusion of a pathway 104 from a photon-generating source 103 (light) to the cathode 100 that is sufficiently transparent at a wavelength that photoexcites the plasmon-resonating nanostructure. Typically, a electrochemical cell is contained within a structure having an exterior wall 110, the exterior wall is preferably a window (a photon-transfer device) that is sufficiently transparent at a wavelength that photoexcites the plasmon-resonating nanostructure. As used herein “sufficiently transparent” means, by way of example, that greater than 25% of the light transmitted in the direction of the cathode 100 by the photon source 103 passes through the window, that is, the window has a 25% transmission. Preferably, the percent transmission is greater than 50%, 60%, 70%, 80%, or 90%. In the absence of an exterior wall, for example when a light source is contained within an electrochemical cell, the pathway is sufficiently transparent if greater than 25% of the light transmitted in the direction of the nanostructure by the photon source reaches the nanostructure.

Preferably, the electrochemical cell includes an oxidant 108 in fluid communication with the cathode 100 and a reductant 109 in fluid communication with the anode 101. An electrical current can be obtained from the electrochemical cell for example by electrically connecting the cathode 100 and the anode 101 to an external circuit 107 by way of electrical leads 105 & 106.

Preferably, the electrolyte 102 in the electrochemical cell is a polymer electrolyte membrane. The polymer electrolyte membrane can be selected from a group consisting of sulfonated polymer membranes (e.g., perfluorosulfonic acid polymer membranes, fluorosulfonic acid polymer membranes, and non-fluoronated sulfonated polymer membranes), acid-base complex membranes, ionic liquid based membranes, inorganic composite membranes, and a mixture thereof.

In embodiments where the electrochemical cell is a hydrogen fuel cell, the reductant 109 is dihydrogen (H₂) and the oxidant 108 is dioxygen (O₂). When the electrochemical cell is a direct methanol fuel cell, as in FIG. 18, the reductant 109 is methanol and the oxidant 108 is dioxygen (e.g., in air). As described above, the plasmon-resonating nanostructure in the electrochemical cell can be a nanoparticle selected from a group consisting of a copper, a silver, and a gold nanoparticle.

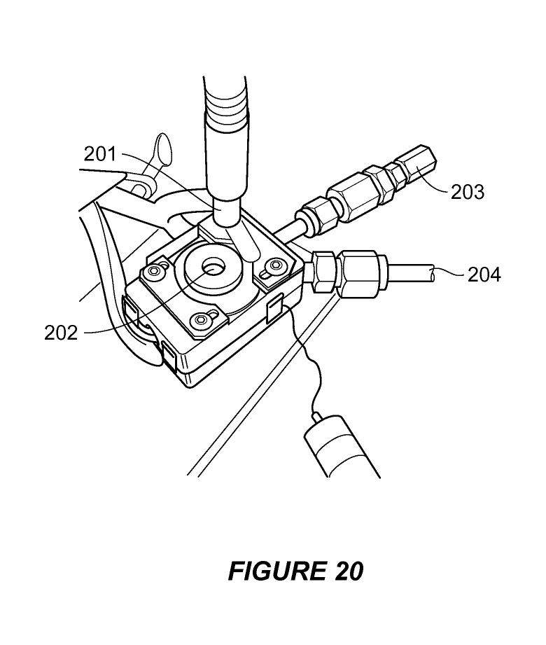

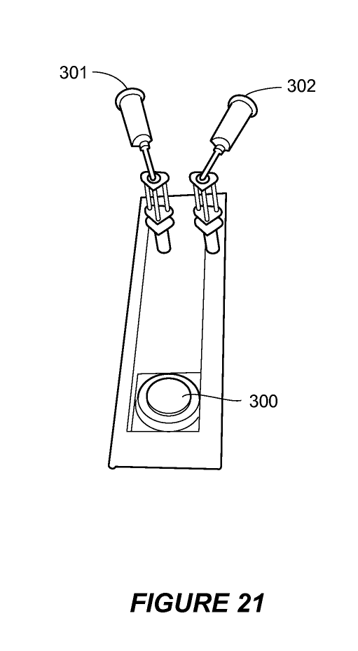

In another embodiment, the plasmon-resonating nanostructure can be included in a catalytic reactor or device. See FIGS. 20 and 21. The reactor or device, as shown in FIGS. 20 and 21, includes a reactant source or line 203, 301 and a product removal pathway or line 204, 302. The reactor or device, as shown in FIGS. 20 and 21, additionally includes a window 202, 300. The reactor or device can further include a light source 201. Preferably, the device includes a plasmon-resonating nanostructure, a support for the plasmon-resonating nanostructure, and a pathway from a photon source to the plasmon-resonating nanostructure sufficiently transparent at a wavelength that photoexcites the plasmon-resonating nanostructure. Optionally, the pathway can be a window 202, 300 that is sufficiently transparent at a wavelength that photoexcites the plasmon-resonating nanostructure. Preferably, the pathway (window) has a percent transmission of at least 25%, 50%, 60%, 70%, 80%, or 90%. In an embodiment where a light source is contained within the device the pathway is sufficiently transparent if greater than 50% of the light transmitted in the direction of the nanostructure by the photon source reaches the nanostructure.

The device also includes an oxidant in fluid communication with the plasmon-resonating nanostructure and, preferably, a reductant in fluid communication with the plasmon-resonating nanostructure. Optionally, the oxidant and the reductant can be mixed prior to placing them in fluid communication with the device. Preferably, the device catalyzes the epoxidation of ethylene, therein the oxidant is dioxygen and the reductant is ethylene. The device can further catalyze the oxidation of carbon monoxide and/or ammonia. In another embodiment, the plasmon-resonating nanostructure included in the device is a nanoparticle selected from a group consisting of a copper, a silver, and a gold nanoparticle.

### EXAMPLES

The following examples are provided to illustrate the invention, but are not intended to limit the scope thereof. The following materials were used to produce these herein reported examples: Ethylene Glycol (J. T. Baker item 9300 with chloride concentration below 0.1 PPM and iron concentration below 0.01 PPM); AgNO₃ (99% purity, Sigma Aldrich cat. No. 209139); Polyvinylpyrrolidone (PVP) 55,000 M.W. (Sigma Aldrich cat. No. 856568); Concentrated HCl; 20 mL glass vials; magnetic stir bars (cleaned with either piranha solution or with Alconox and successive sonication in water, ethanol and acetone); and a syringe pump.

### Synthesis of Silver Nanocubes

5 mL Ethylene Glycol and a magnetic stir bar were added to a 20 mL vial and submerged in an oil bath heated to 140-145° C. on a stirring hotplate. The cap to the vial was loosely placed on top to allow boiling off of vapors from any contaminant solvent. After 1 hr of heating, 100 μL of 30 mM HCl in ethylene glycol was added to the hot ethylene glycol. After 5-10 minutes, 3 mL of 0.1 M AgNO₃ in ethylene glycol and 3 mL of 0.15 M PVP (in terms of repeating unit) in ethylene glycol were added to the heated vial using a syringe pump at a rate of 0.75 mL/min. After this addition, the cap was loosely placed back on the vial, (1 turn just to secure the cap). The solution was allowed to stir for about 24 hrs. After 24 hours the cap on the vial was tightened such that the vial became airtight. Over 2-3 hours a series of color changes were observed resulting in a thick tan/ocher colored solution. (The size of the particles can be tuned by changing the amount of HCl added to the system). This procedure yielded cubes of ˜60-70 nm edge length. Decreasing the volume of 30 mM HCl added to the synthesis to 60 μL produces cubes of about 110 nm edge length.

Synthesis of Copper Nanoparticles

An aqueous solution (900 μL) of 0.1 M Cu(NO₃)₂ was added to a mixture (10 mL) of n-heptane and 16.54 wt. % of polyethylene glycol dodecyl ether (average Mₙ˜362, Brij 30, available from Sigma-Aldrich) at room temperature (20-25° C.). The copper admixture was stirred for 15 minutes, then an aqueous solution (900 μL) of 1 M hydrazine was added dropwise (˜25 μL). The reaction vessel was then tightly closed and stirred for about 18 hours to yield a copper nanoparticle microemulson.

### Preparation Of Supported Nanocrystals

SILVER—The silver nanoparticles (about 0.025 g) were dispersed in a 5 mL ethanol solution, 0.1 g of α-Al₂O₃ was added to the solution and the mixture was sonicated for 1 h. The solution was then dried yielding silver supported nanocrystals.

SILVER Pretreatment—The supported nanocrystals were loaded into a Harrick-type high temperature reaction cell with a 1 cm² window, allowing direct visible light illumination of the supported nanocrystals. The reaction cell was flushed with 20 sccm O₂ and 60 sccm N₂ for 2 hours at 220° C. (oxygen pre-treatment). In the case of ethylene epoxidation, after the oxygen pre-treatment, the reaction cell was flushed with 20 sccm of ethylene in addition to the O₂ and N₂ at 220° C. (reactant pre-treatment). The reactant pre-treatment was continued until reaction products stabilized, as determined by quadrupole mass spectrometry. In the case of CO, NH₃, or propylene (etc.) oxidation, the procedure is identical to ethylene epoxidation except the appropriate reactant is introduced into and following the pre-treatment.

COPPER—A sufficient amount of support material, SiO₂ (surface area 390 m²/g, Aldrich), was added to the copper nanoparticle microemulsion, described above, to yield a final composition of approximately 2 wt % Cu/support material (SiO₂). Following the addition of the support material, the suspension was stirred for about 1 hour, then ethanol was added and the surfactant was removed. The copper nanocrystals supported on the support material was collected by centrifugation at 4500 rpm for 30 minutes, then dried under argon at room temperature.

COPPER Pretreatment—The supported copper nanocrystals were then added to a packed bed reactor (reaction cell), where 15 mg of silica beads were added to the bottom of the catalyst bed then 20 mg (total weight) of the supported copper nanocrystals (2 wt% Cu/SiO₂) was loaded on top of the silica beads. The reaction cell was flushed for 2 hours with 5% hydrogen (remaining helium) at a total flow rate of 100 cm³/min at 230° C. (hydrogen pre-treatment).

Oxidation Over Silver:

### Example 1

Initial temperature dependent photothermal experiments were conducted to examine the effect of visible light illumination on the activity and selectivity for the ethylene epoxidation reaction, CO oxidation reaction and NH₃ oxidation reactions. At each temperature the catalyst was allowed 15 minutes to reach steady state under dark conditions followed by 15 minutes of visible light illumination, followed by 15 minutes in the dark to assure that the activity returned back to the initial dark baseline. The enhancement is calculated as the total rate under visible light illumination divided by the pure thermocatalytic rate with no illumination. In the case of ethylene epoxidation, the selectivity to ethylene oxide as well as the ethylene oxide yield as a function of temperature is also examined. The visible source used in all the experiments is a broad band white light source with an intensity of 50 mW/cm² and a maximum output at 580 nm.

Ethylene epoxidation:

C₂H₄+½O₂→C₂H₄O (EO) or (C₂H₄+3O₂→2CO₂+2H₂O)

In these experiments m/z 44 accounts for both products ethylene oxide and CO₂ and is used as a measure of overall activity. The ratio between m/z 43 and m/z 44 is used to calculate selectivity based on a calibration of the relative strengths of these peaks for EO.

CO Oxidation:

CO+ 1/2O₂→CO₂

In these experiment m/z 44 accounts for the product production and is used to calculate enhancements.

NH₃ Oxidation:

NH₃+O₂→N₂+N₂O+NO+NO₂+H₂O (non-stoichiometric)

In this reaction a number of masses were monitored N₂ (14), N₂O (44), NO (30), NO₂ (46). The selectivity was not tabulated.

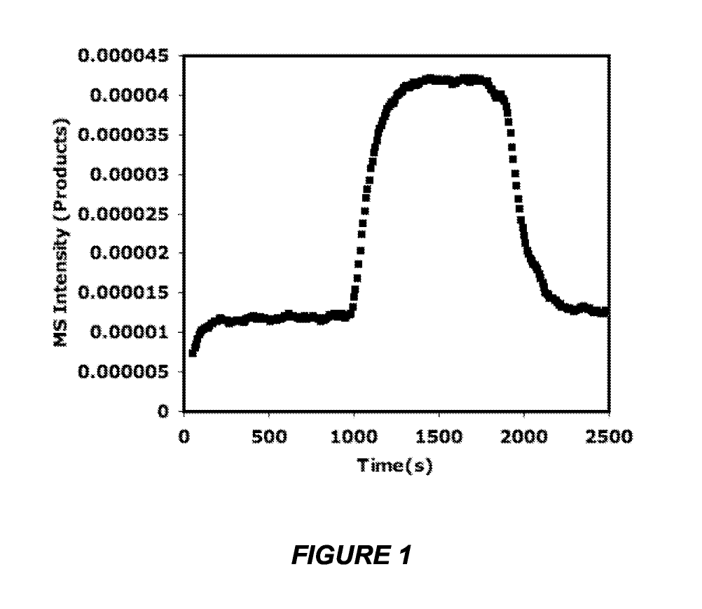

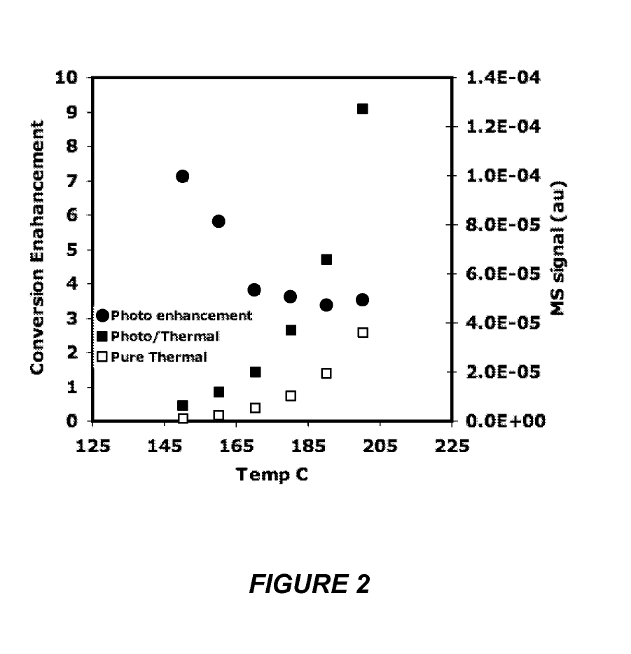

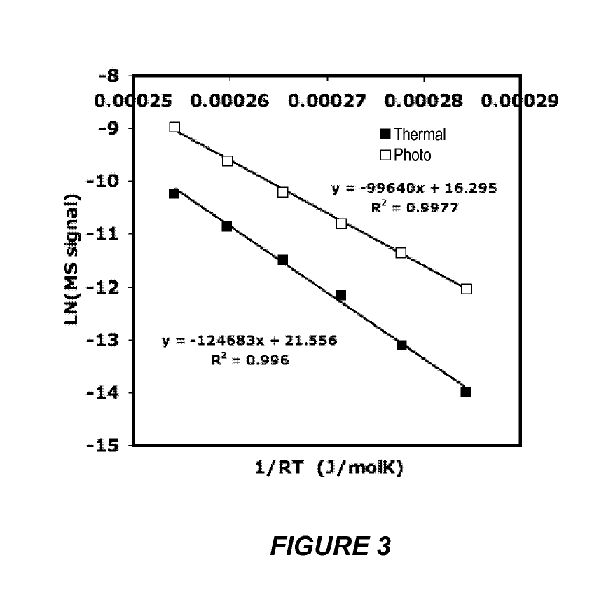

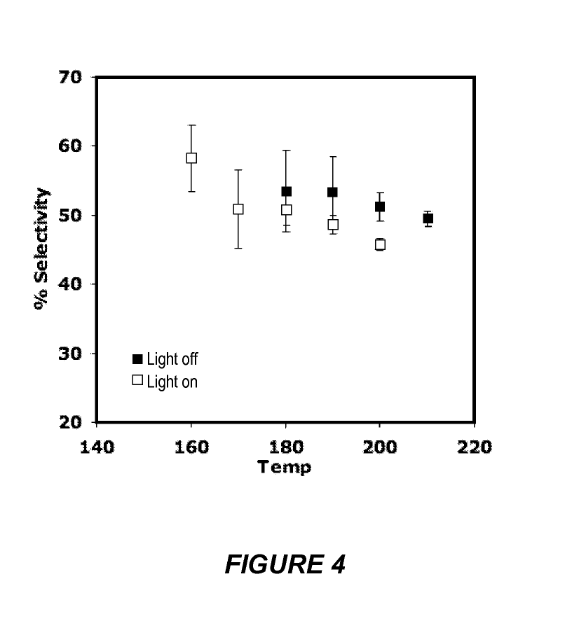

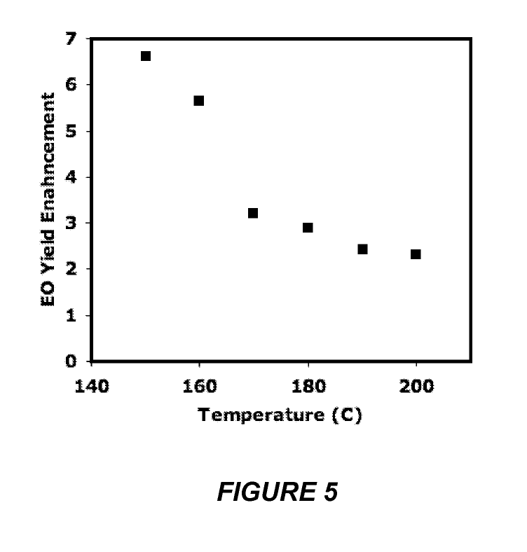

FIG. 1 shows the mass spectroscopy signal (m/z 44 which accounts for both ethylene oxide and CO₂ production, is a measure of conversion) as a function of time for the ethylene epoxidation reaction. In this case the light was turned on at approximately 950 seconds, showing a large increase in ethylene conversion (about 4 fold at 180° C.), and turned off at about 1800 seconds. This is characteristic for all the photo-thermal processes described herein, i.e. a marked increase in activity as soon as the visible light is introduced. FIG. 2 shows the photo-enhancement as a function of temperature for ethylene epoxidation as well as the pure thermal and the photothermal rates as a function of temperature. The enhancements in these experiments range from 8 fold at low temperatures to about 3 at high temperatures. FIG. 3 shows a kinetic analysis of both the thermal and photothermal rates. The activation barrier for the photothermal reaction is significantly less than for the pure thermal case. FIG. 4 shows the measured selectivity to ethylene oxide as a function of temperature showing only a minimal change (3-5%) in selectivity in the photocatalytic reaction. FIG. 5 shows the ethylene oxide yield (selectivity*conversion) enhancement as a function of temperature showing a substantial yield enhancement across the entire range of temperatures.

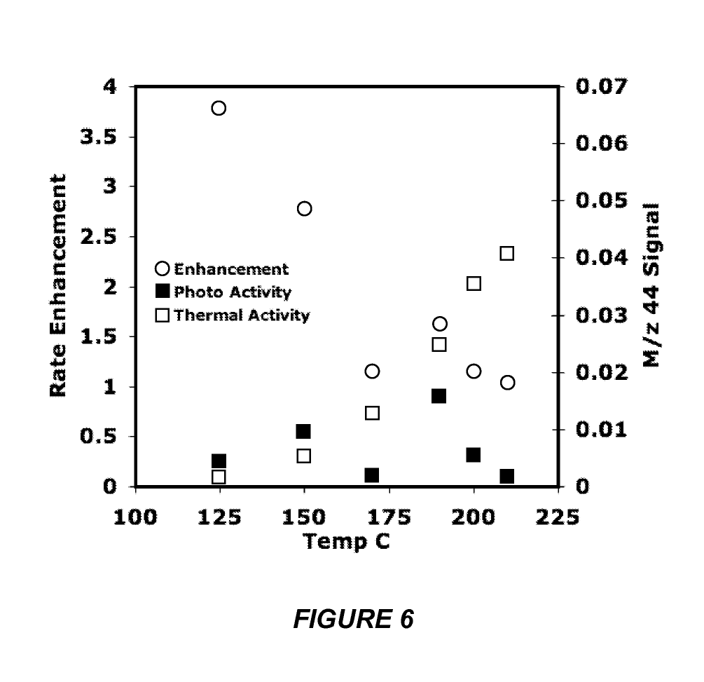

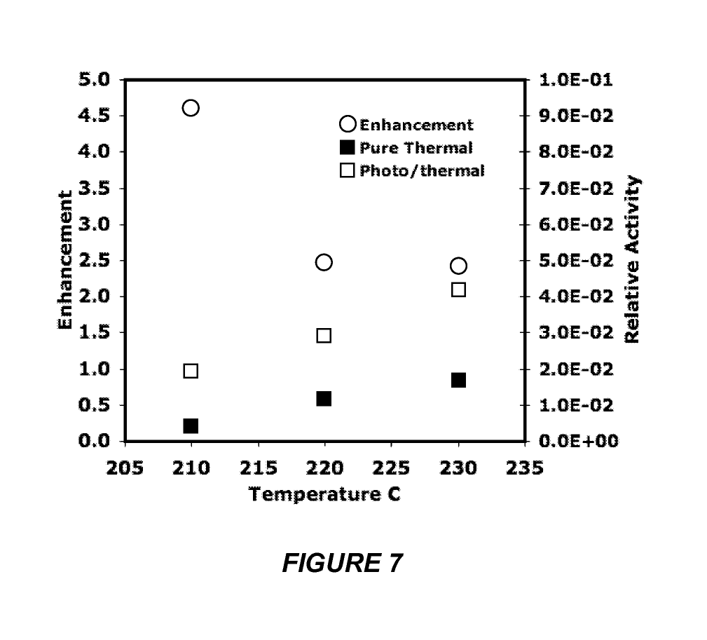

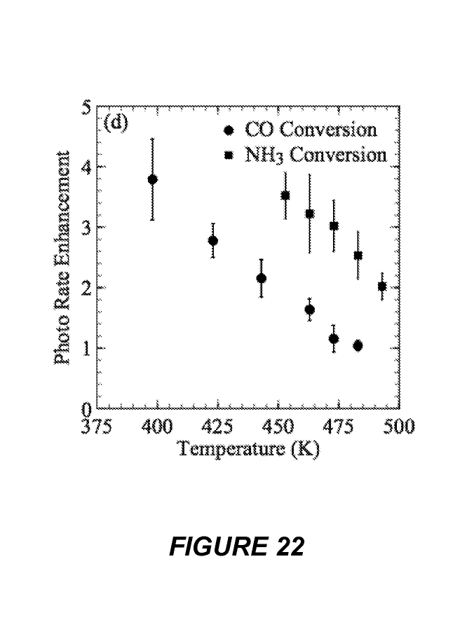

FIGS. 6 and 7 show the rate enhancement, thermal rate and photothermal rate for CO and NH₃ oxidation, showing similar trends to ethylene epoxidation and significant enhancements. FIG. 22 compares the rate enchancement for both the CO and NH₃ oxidation as a function of temperature, calculated by dividing the photo-thermal rate by the pure thermal rate, error bars are the standard deviation of the systematic errors in the collection of mass spectrometer data.

### Example 2

### Intensity And Wavelength Dependence Of Ethylene Epoxidation Over Silver

### Intensity Dependent Photothermal Tests

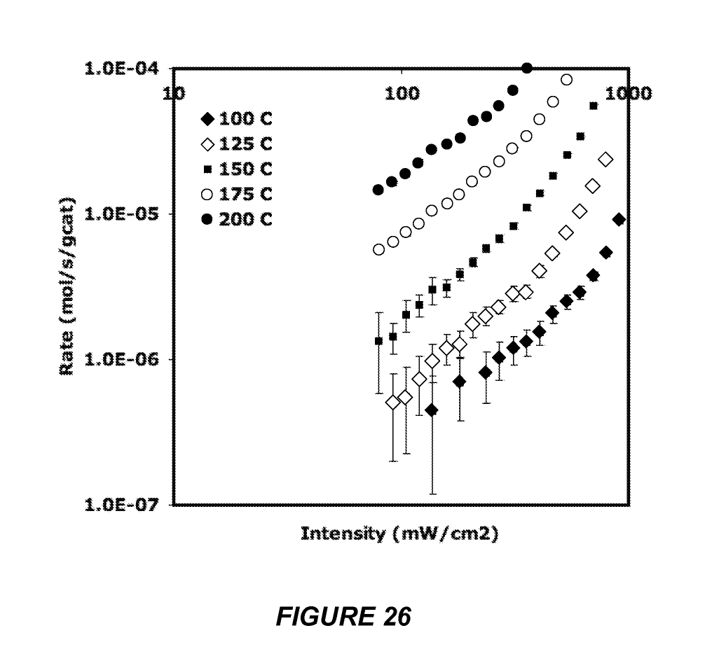

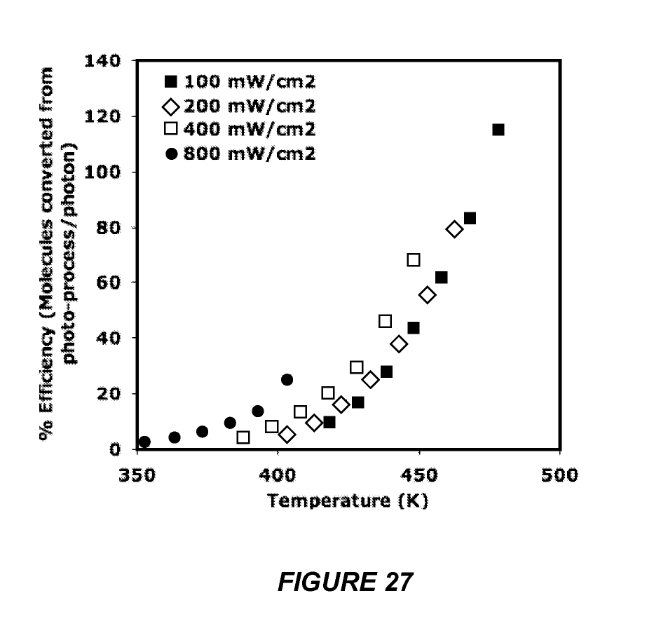

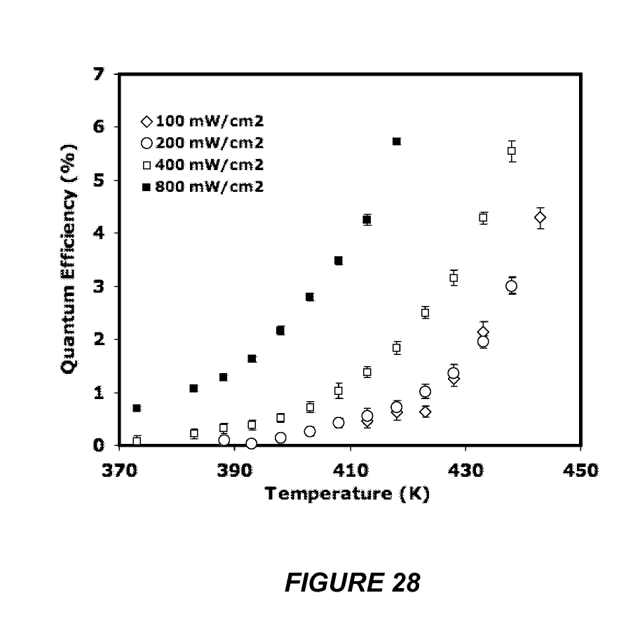

The mechanism of photocatalytic rate enhancement and the effect of source intensity on the photo-enhancement were examined by intensity dependent experiments. The intensity was varied by controlling the power input to the source but also could have been controlled by filters or other means. FIG. 25(b) indicates a direct (linear) correlation between the photocatalytic activity and the source intensity up to 250 mW/cm². This linear dependence on source intensity is indicative of an electron driven process. FIG. 26 shows the rate of ethylene epoxidation over silver nanocrystals vs. incident photon intensity as a function of reactor temperature. Unlike FIG. 25(b), the high intensity photo-catalytic process is indicative of a multi-electron driven process. FIG. 26 shows the conversion efficiencies for ethylene to ethylene oxide as a function of temperature for different incident photon intensities. FIG. 28 shows the conversion efficiency per quantum of light (reported as a quantum efficiency) for ethylene to ethylene oxide as a function of temperature for different incident photon intensities. These results indicate that the efficiency of the ethylene to ethylene oxide reaction can be increased by increasing either the temperature or the photon intensity. For example, from FIG. 27, the percent efficiency can be increased from about 5% (200 mW/cm² at 410 K) to about 25% by alternatively increasing the temperature to 440 K (at 200 mW/cm²) or increasing the photon intensity to 800 mW/cm² (at 410 K). The ability to achieve high efficiencies at low temperatures can promote catalyst stability and will provide enhanced catalyst lifetimes.

### Wavelength Dependent Studies

To further examine the mechanism of enhancement, wavelength dependent studies were performed using a series of seven long pass filters, which allow different wavelength ranges of light to hit the catalyst surface. The rate enhancement was monitored as a function of long pass filter. FIG. 25(a) indicates a direct correlation between the wavelength dependence of the silver catalyzed photocatalytic activity for ethylene epoxidation and the wavelength dependence of the silver plasmon intensity. The normalized photo-catalytic ethylene epoxidation values were calculated by subtracting the thermal rate (light off) from the photo-thermal rate (light on) for each filter cutoff wavelength and then dividing by the photo-rate with no filter. Error bars in the plots represent the standard deviation of the systematic errors in the collection of mass spectrometer data.

There are two plausible mechanisms of photocatalytic activity on plasmonic silver particles: localized plasmon heating and energetic electron donation. Regarding the possibility of plasmonic local heating as a mechanism for the observed photocatalytic activity, the magnitude of plasmonic nanoparticle heating is a function of the source intensity, size of the nanoparticle and the local environment. A simple model can be developed to describe the steady state temperature change of an illuminated nanoparticle, based on conduction of heat from a 60 nm cube that is producing a steady state flux.

This model shows that the temperature change due to plasmonic heating is linear with respect to the intensity of the illumination source. Because of this linear relationship, the rate enhancement as a function of source intensity should exhibit an exponential dependence, as the rate of thermally driven reactions is known to follow the Arrhenius rate equation. The intensity dependence of the photocatalytic rate was determined by varying the source intensity from 5 mW/cm² to 50 mW/cm², showing a linear dependence between the intensity and photocatalytic rate. A model prediction, developed with inputs from the purely thermal experimental results: temperature, enhancement and activation barrier, predicts an exponential increase in photoactivity as a function of intensity, which disagrees with the low intensity experimental results.

To further verify that the photoreactivity is plasmon based and further test the validity of the local heating mechanism, wavelength dependent experiments were performed using long pass filters. The results of these experiments along with model predictions based on the nanoparticle heating model and model where photocatalytic rate depends linearly on plasmon concentration. In these models the relative heating potential and the linear photon driven reactivity potential are simulated by normalizing the energy dependent source intensity by the energy dependent plasmon resonance intensity as a function of long-pass filter energy. The figure shows a good agreement with the linearly dependent rate-intensity model, indicating that this is a plasmonically driven effect, not due to local plasmonic heating.

Semiconductor photocatalytic process also commonly exhibit a similar linear dependence, indicating the photocatalytic enhancement process is very similar to conventional semiconductor photochemistry where photoexcited electron holes pairs drive the chemical transformation, but in this case some thermal energy or high intensity photon fluxes are needed to overcome the activation barrier.

### Example 3

### Isotopic Labeling Experiments

The mechanism of photocatalytic activity was examined by monitoring the effect of labeled ¹⁸O on the pure thermal and photothermal catalytic reactions. ¹⁸O was introduced for 10-15 minutes to allow the system to reach steady state and the quantity of ¹⁸O based products (m/z 46 and 48) were monitored. FIG. 25(c) shows the results of the effect of switching from ¹⁶O₂ (at least 99% ¹⁶O₂) to ¹⁸O₂ (at least 99% ¹⁸O₂) in the photo-thermal ethylene epoxidation with a silver catalyst; a 16% decrease in reaction rate. This comparison the isotopic effect for the analogous thermal process showed a 5% decrease in reaction rate when ¹⁸O₂ was used.

We believe the enhancement mechanism is similar to one that has been previously found to play a role in femtosecond laser induced desorption/reaction experiments on metallic surfaces is the hot electron induced population of unpopulated adsorbate antibonding orbitals. Femtosecond laser induced photochemistry experiments show the possibility for activating chemical reactions that cannot be performed using only thermal energy input. Although these systems have achieved considerable attention scientifically, it is not practical to use energy-intensive femtosecond lasers to drive reactions. Stemming from these studies it has been shown that reactions driven by energetic electrons on metal surfaces exhibit significant isotope effects that are not present in thermal reactions. The isotopically labeled molecules must be involved in the rate-determining step to exhibit the strong effect on photoactivity.

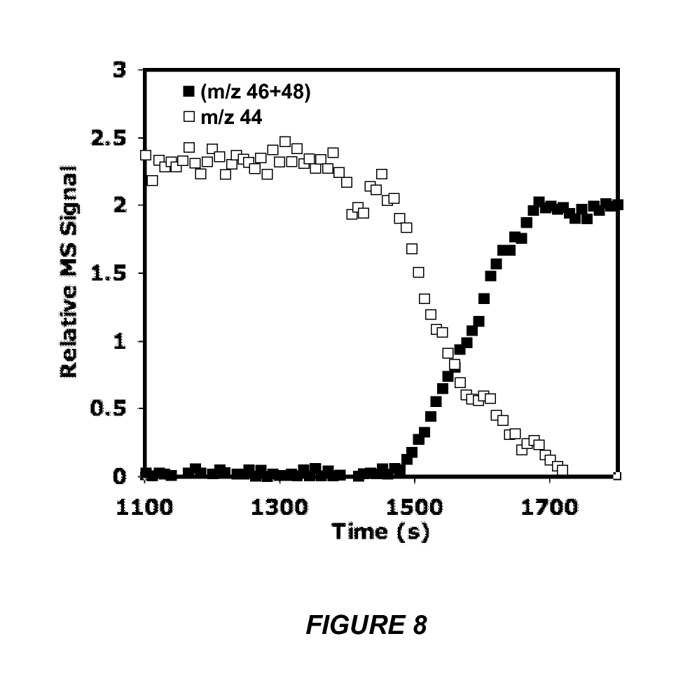

We have performed steady-state activity experiments with isotopically labeled oxygen as the reactant (O₂ dissociation is known to control the rate of ethylene epoxidation at low temperatures). The steady state activity of the ¹⁸O and ¹⁸O based processed are presented in FIG. 8. The isotope effect that is plotted is simply the steady state activity of ¹⁸O based process divided by the steady state activity of ¹⁸O based processes. This shows that with the light off there is very little isotope effect, where as with the light on there is a significant drop in activity when ¹⁸O is introduced.

### Example 4

### Manufacture Of A Catalytic Reactor

Reactor cells were fabricated using 100 mm borofloat glass wafers and (100) silicon wafers. Flow channels were etched 50 km into the silicon wafer using an STS Pegasus deep reactive ion etcher with a photoresist mask. Thermal isolation was provided by backside etching the silicon using a MA/BA-6 for backside exposure alignment and etched with an STS Pegasus deep reactive ion etcher. An insulating dielectric layer (silicon dioxide, 100 nm) was deposited on the silicon channels using a GSI plasma enhanced chemical vapor deposition (PECVD) instrument. Access holes to the reactor were created by electrochemically drilling holes into the borofloat glass wafer. Both the silicon and glass wafer were then cleaned using piranha solution (3:1 H₂SO₄:H₂O₂), sonicated in both acetone and 2-proponal, then surface activated with nitrogen plasma using an nP-12 instrument. About 20 μL of catalyst were deposited into the reaction area and dried. The glass and silicon wafers were then bonded using an SB-6E anodic bonder at 250° C. and −1000V. Bonded devices were finally diced and then fitting were connected using optical adhesive, two part epoxy and EFD precision tips.

Oxidation over Copper:

### Example 5

### Propylene Epoxidation

Propylene was epoxidized by supported copper nanocrystals by flushing the reaction cell with a gas composition that includes 20% propylene, 20% oxygen and 60% of an inert gas (e.g., helium) at a total flow rate of 100 cm³/min. The reactants and products were analyzed using a gas chromatograph (Varian CP 3800) equipped with thermal conductivity and flame ionization detectors. All of reported results were measured under steady state reaction conditions.

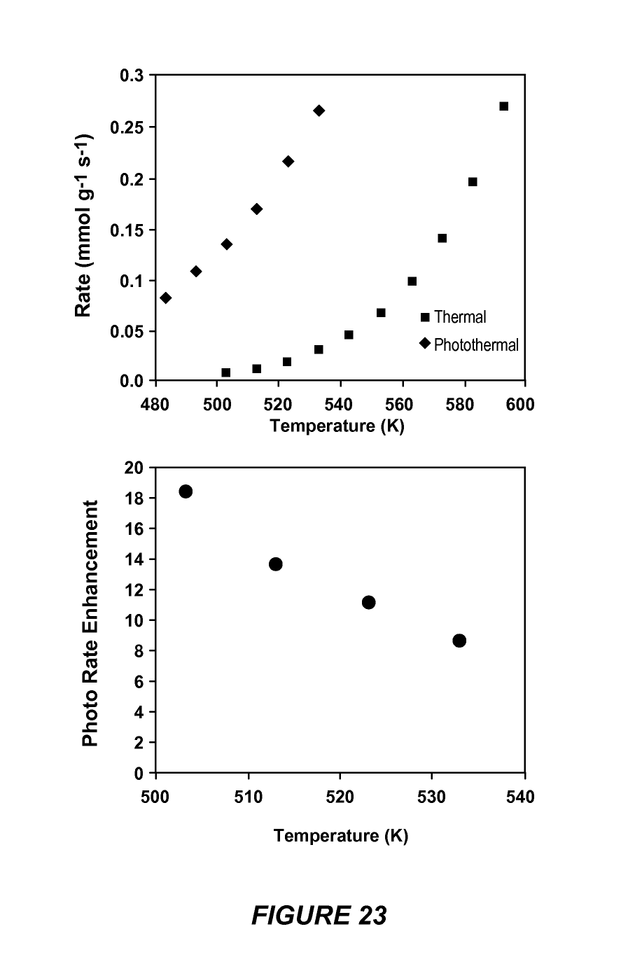

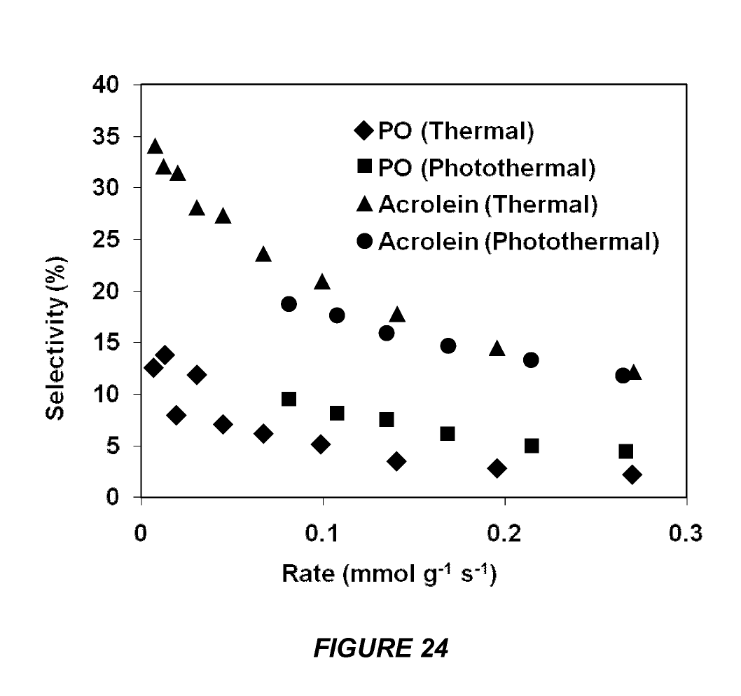

FIG. 23(a) shows the rate of propylene epoxidation the 2% Cu/SiO₂ catalyst under thermal and photo-thermal conditions as a function of temperature. FIG. 23(b) shows the photo rate enhancement for the results presented in FIG. 23(a). FIG. 24 shows the product distribution (selectivity) for propylene oxide (PO) and acrolein at thermal and photo-thermal conditions. These results show that the selectivities at thermal and photo-thermal conditions are approximately the same.

The foregoing description is given for clearness of understanding only, and no unnecessary limitations should be understood therefrom, as modifications within the scope of the invention may be apparent to those having ordinary skill in the art.

1. A method comprising:
supplying an oxidant having a π-antibonding orbital to a surface of a plasmon-resonating nanostructure;
exposing the plasmon-resonating nanostructure to photons at a wavelength sufficient to photoexcite the plasmon-resonating nanostructure; and
reducing the oxidant at a rate about 1.1 to about 10,000, times the rate of reduction of the oxidant under the same conditions but in the absence of the photons.

2. The method of claim 1, wherein the step of
reducing the oxidant comprises reducing the oxidant at a temperature below a predetermined thermodynamic barrier.

3. The method of claim 2, further comprising supplying and oxidizing a reductant at the temperature below the predetermined activation temperature.

4. The method of claim 3, wherein the reductant is an alkene.

5. The method of claim 4, wherein the alkene is selected from the group consisting of ethylene, propylene, and butylene.

6. The method of claim 3, wherein the reductant is a material selected from the group consisting of hydrogen, methanol, and ammonia.

7. The method of claim 1, wherein the plasmon-resonating nanostructure is present on a support.

8. The method of claim 7, wherein the support is one of silica and alumina.

9. The method of claim 1, wherein reducing the oxidant produces an oxidation product selected from a group consisting of water, ethylene oxide, propylene oxide, acrylonitrile, propenal, acrylic acid, carbon dioxide, nitrous oxide, nitric oxide, nitrogen dioxide, and mixtures thereof.

10. The method of claim 1, wherein the oxidant is selected from the group consisting of dioxygen (O₂), dinitrogen (N₂), nitrous oxide and ozone.

11. The method of claim 10, wherein the oxidant is dioxygen (O₂).

12. The method of claim 1, wherein the plasmon-resonating nanostructure catalyzes the reduction of the oxidant.

13. The method of claim 1, wherein the plasmon-resonating nanostructure comprises a nanoparticle selected from the group consisting of copper, silver, gold, and alloys thereof.

14. (canceled)

15. The method claim 2, wherein the temperature at which the oxidant is reduced is about 20° C. to about 100° C. below the predetermined activation temperature.

16. An electrochemical cell comprising:
an electrolyte;
a cathode comprising a plasmon-resonating nanostructure;
an anode separated from the cathode by the electrolyte; and
a photon-transfer device that is sufficiently transparent at a wavelength that photoexcites the plasmon-resonating nanostructure.

17. The electrochemical cell of claim 16 further comprising an oxidant in fluid communication with the cathode; and a reductant in fluid communication with the anode.

18. (canceled)

19. (canceled)

20. (canceled)

21. The electrochemical cell of claim 20, wherein the electrolyte is a polymer electrolyte membrane selected from the group consisting of sulfonated polymer membranes, acid-base complex membranes, ionic liquid based membranes, inorganic composite membranes, and mixtures thereof.

22. A device comprising:
a plasmon-resonating nanostructure;
a support for the plasmon-resonating nanostructure; and
a photon-transfer device that is sufficiently transparent at a wavelength that photoexcites the plasmon-resonating nanostructure.

23. The device of claim 22 further comprising an oxidant and a reductant in fluid communication with the plasmon-resonating nanostructure.

24. (canceled)

25. (canceled)

26. (canceled)

27. (canceled)

28. A method comprising:
supplying an oxidant having a π-antibonding orbital to a surface of a plasmon-resonating nanostructure;
exposing the plasmon-resonating nanostructure to photons at a wavelength sufficient to photoexcite the plasmon-resonating nanostructure; and
reducing the oxidant at a temperature below a predetermined thermodynamic barrier.
